(19) United States
(12) Patent Application Publication
(10) Pub. No.: US 2017/0300111 A1
(43) Pub. Date: Oct. 19, 2017

(54) SURGICAL DEMONSTRATION SYSTEM AND MODULAR SIMULATION TRAINING TOOL INCLUDING ARTIFICAL TENDONS

(71) Applicant: PROSURGICAL LLC, Green, OH (US)

(72) Inventors: Zac Price, Uniontown, OH (US); Gregory Thomas Hartz, SR., Akron, OH (US)

(21) Appl. No.: 15/643,041

(22) Filed: Jul. 6, 2017

Related U.S. Application Data
(63) Continuation-in-part of application No. 15/474,609, filed on Mar. 30, 2017.
(60) Provisional application No. 62/316,992, filed on Apr. 1, 2016.
(60) Provisional application No. 62/359,107, filed on Jul. 6, 2016.
(60) Provisional application No. 62/520,858, filed on Jun. 16, 2017.

(51) Int. Cl. G06F 3/01; A61B 34/10

(52) U.S. Cl. CPC G06F 3/011; G06F 3/016; A61B 34/10; A61B 2034/105; G05B 2219/45123

(57) ABSTRACT

The disclosure provides a modular surgical demonstration system and training tool that has a base member with a plurality of fixture devices which are removably supported thereon in an engagement that permits demonstration on an anatomical portion of the fixture. The tool includes at least one surgical training fixture configured to hold both hard and soft surgical tissue simulation during training procedures. In a further embodiment, the soft tissue includes ligament or tendon simulation member which mimic the look and feel of natural tendon.

### CROSS-REFERENCE TO RELATED APPLICATIONS

This application claims priority under 35 USC §120 to U.S. Provisional Patent Application Ser. No. 62/316,992, filed Apr. 1, 2016 and U.S. Provisional Patent Application Ser. No. 62/359,107 filed Jul. 6, 2016 and U.S. Provisional Patent Application Ser. No. 62/520,858 filed Jun. 16, 2017 and is a Continuation in Part application of U.S. patent application Ser. No. 15/474,609, filed Mar. 30, 2017, all herein fully incorporated by reference.

### FIELD OF THE INVENTION

A modular surgical demonstration system and training tool that has a base member with a plurality of fixture devices which are removably supported thereon in an engagement that permits demonstration on an anatomical portion of the fixture. The tool includes at least one surgical training fixture configured to hold both hard and soft surgical tissue simulation during training procedures. In a further embodiment, the invention also includes artificial ligaments that are used for training and demonstration purposes.

### BACKGROUND OF THE INVENTION

Medical sales and distribution personnel often provide an introduction for medical devices to both new and established medical practitioners. The setting for this introduction can vary from short in office meetings to seminars in continuing education settings to large industry conferences. Often the technique for implanting a new device is illustrated using cadavers. While cadaver work provides the acting surgeon with an experience that recreates an actual surgery, it is expensive and severely limits the amount of experience that practicing surgeons can get, and may not even be available to surgeons in training. Moreover, since it involves biological matter, the environment for the surgery and the disposal of the cadaver must be closely monitored, adding to the expense and inhibiting the circumstances under which the device and technique can be demonstrated and tested. For these reasons, the marketing of new devices is also severely limited. Thus, there is a real need for a system for demonstrating, providing information, training and even perfecting surgical technique that is free from the constraints associated with cadaver work.

In addition to the need for convenient hands-on informational systems, there is a need for tools that can also be used for training purposes. Surgeon training is typically based on an apprenticeship-type model whereby residents or untrained surgeons learn various surgical techniques by assisting or otherwise viewing experienced surgeons conducting the relevant technique. The resident or untrained surgeon typically views the procedure multiple times and increases their hands on participation during each experience so that over time they gain experience in a hands-on manner with every aspect of the procedure from start to finish. This process has shown to be an effective teaching tool; however it is known to be extremely costly and time-consuming. Therefore, there is a need for an improved surgical training device or methodology that allows residents or untrained surgeons to practice various surgical procedures outside of the actual operating room to gain familiarity and experience with the procedure prior to entering the operating room. Further, there is a need for a device to allow surgeons to demonstrate and teach surgical procedures to residents without requiring an actual patient/cadaver and an operating room. A device such as this will provide the resident or untrained surgeon the ability to view and learn surgical procedures while requiring much less training in the actual operating room before they are able to conduct a procedure on their own. This need extends to demonstrations and seminars of lessons that relate to procedures done on tendons. In a further embodiment, the present invention includes artificial tendons that can be used with the demonstration system to replicate surgical procedures involving tendon graft or tendon repair with artificial tendon simulations that avoid the issues presented by biological tendons or tendon substitutes.

### SUMMARY OF THE INVENTION

Embodiments of the invention provide a surgical demonstration, marketing and training system and tools of all of these facets of the medical implant business. Ideally, the system is modular (meaning that a number of different procedures and anatomies are demonstrated, for example by providing removable fixtures each representing various demonstrations) and can support a number of device platforms. It is quick to set up and extremely user friendly. It is easily transported. It is designed to allow the actual manipulation of a medical device in a manner which mimics the surgical use, and which may even provide for a training environment which is superior for the purpose of illustrating the use and best practice of a medical device. The system generally includes a base that supports a series of fixtures so as to enable a demonstration of, and work with medical implants using the fixtures as representations of the biological environment. The interaction of the base member and the fixture retains the anatomical feature at an optimal position to demonstrate and provide access to the anatomical feature, but sufficiently securely to inhibit rotation or dislodgement from the base during manipulation and sufficiently easily to provide an assembly that is easily and quickly assembled and in which various fixtures can be switched out for demonstration purposes.

Thus the system includes a base member and a series of modular fixtures having anatomical features. The fixtures and base member include a mating relationship so that the fixtures can be mounted in the base member so as to optimize the position of a work surface on the fixture. The positioning allows the demonstrator to stand away and allow the user (i.e. the medical person or surgeon in training) to have a clear and unobstructed view and work space to learn and practice technique. This mating relationship inhibits movement of the fixture relative to the base during use, but is releasable so that the fixtures can be quickly and easily assembled in the base, for example, by a sales person who can continue to discuss the device without having to concentrate on the assembly process. Thus, it is advantageous if the mating relationship is a press fit, with either the fixture or the base including a retaining means, such as a recess having side walls that form an angle relative to a reference plane, such as the top surface of the base, or a plane of a table which supports the system. It should be understood that more complex interactions of the base and fixtures can also be used, for example, a snap lock relationship in which the fixture snaps into position and a ring is press fit into a corresponding recess in the base to more securely hold the fixture in place.

Ideally for a press or friction fit engagement, the side wall of the fixture recess in the base member or alternatively in the base boss recess in the fixture base is nominally vertical relative to the reference plane, and by that it is meant that the wall forms a 90° angle +/−5°, and preferably +/−3°, and more preferably +/−2°. Thus, the wall can cant inward or outward slightly to ensure that the press fit will be sufficiently tight over time and use. Likewise, the mating piece, either the fixture or the base retainer has a nominally vertical sidewall of a corresponding outline and angle. It is possible that the mating piece has a slightly different angle, such as a side wall that expands upward on the fixture relative to a recess in the base member in order to assure a tight fit which will withstand from 0.5 to 15 Newtons of upward force to disassemble the fixture from the base member.

In a first embodiment, the invention provides the base, a first fixture receptacle having a recessed portion defined by a substantially planar top surface of the base surrounding the recessed portion and including and vertical recess wall or walls depending on the outline of the recess, a plurality of second fixture receptacles defining cylindrical bores formed partially through the fixture base, and at least one knot tying station comprising a through hole formed between a top and side surface of the base member. The tool may further include a surgical training fixture that has a fixture base member sized to be frictionally received in the recessed portion of the first fixture receptacle, and a surgical training fixture joint portion attached to the fixture base member and extending therefrom, the joint portion being sized and shaped to approximate a specific surgical area. In addition, the fixtures may include posts having clamps for simulated tendon grafts, and in a further embodiment of the invention, artificial simulated tendon grafts are provided which replicate the “look and feel” of human tendons in order to best simulate the actual procedure which is performed on the tendons.

Embodiments of the disclosure may further provide a surgical demonstration system and training tool that has a weighted or clamped rectangular base member having a centrally positioned rectangular shaped first fixture receptacle (including a space for marketing material), the first fixture receptacle being recessed into the rectangular base member and defined by a substantially planar bottom surface and vertical recess wall. The tool may also have at least one circular fixture receptacle positioned near a distal end of the rectangular base, each of the circular fixture receptacles further optionally having a plurality of cylindrical holes formed partially into the rectangular base to support posts that include clamps for tendon simulation. The tool may further have a surgical training fixture having a fixture base sized to be frictionally received in the recessed portion of the rectangular shaped first fixture receptacle, and a surgical training fixture surface attached to the fixture base member and extending therefrom, the fixture surface being sized and shaped to approximate a specific surgical area, specifically including, but not limited to a proximal humerus, a distal femur, a proximal tibia, a foot or portions thereof, and a shoulder capsule.

Embodiments of the disclosure may further provide a surgical training and demonstration tool that includes an oblong, i.e. rectangular or modified rectangular base member having a centrally positioned oblong (rectangular) shaped first fixture receptacle, the first fixture receptacle being recessed into the rectangular base member and defined by a substantially planar bottom wall and substantially vertical recess wall. The tool may further include at least one round (i.e. circular) fixture receptacle positioned near a distal end of the rectangular base, each of the circular fixture receptacles having a plurality of cylindrical holes formed partially into the rectangular base, and a surgical training fixture having a fixture base sized to be frictionally received in the recessed portion of the rectangular shaped first fixture receptacle. The side walls may include features such as texturing or texturing to increase a frictional relationship with a mating surface. The tool may further include a convexly rounded member or pair of members, such as a dome shaped or a semi spherical surgical training fixture surface attached to the fixture base member and extending therefrom, the fixture surface being sized and shaped to approximate a specific surgical area, and a daisy or clover shaped tissue specimen attached to an apex of the semi spherical surface, the clover shaped tissue specimen having a central portion including a central attachment member, such as a button or clip, and a plurality of petal-shaped tissue flaps extending therefrom and wherein the tissue flaps are separated by slits extending toward the center of the tissue specimen. These slits extend more than ⅓ of the distance radially into the tissue specimen, and preferably more than ½ of the distance. The shape includes opposing linear slits, and a pair of terminal notches which help to define a plurality of petal members wherein the ends include front and back rounded tabs each flanked on either side by radially asymmetrical petal members which have a rounded edge facing the respective front or back petal, and a rectilinear angled lateral edge facing the slit.

Of course, it should be understood with respect to the mating relationship that either the fixture base or the base member can include the recess which retains the mating member, and more complicated relationships can also be included, such as a recess that includes a flange or boss so that the fixture base of base member is supported by the corresponding piece on both an interior and exterior side wall. In addition, the mating relationship can include a detent or friction member with the base or the fixture base, a biasing member, such as a spring detent, leaf spring, ring or plunger. Ideally the mating relationship will withstand a force of from 0.25-15 Newtons of straight pulling force, and more preferably from 0.5-10 Newtons, and most preferably from 0.75 to 5 Newtons. In addition, the mating relationship will resist rotational forces and pivoting forces which could cause the fixture to be insecure within the base during use. This relationship depends on the relationship between the fixture base and the retainer within the base and the relative fit of the two. Ideally, in the case in which the fixture base sits in a recess in the base, the fixture base has a depth of from 5-30 millimeters, and preferably from 10-20 millimeters and the recess in the base is of a corresponding depth. Also the recess if preferably radially symmetrical, and is ideally a circle or a modified circular shape, such as a torx shape. A useful size is diameter is from 15-80 millimeters, and preferably from 30-60 millimeters, where a useful dimension for the base is from 60-150 millimeters wide, and preferably from 80-120 millimeters wide, from 15 to 60 millimeters in depth, and preferably from 25 to 40 millimeters in depth and from 200 to 400 millimeters in length, and preferably from 220 to 300 millimeters in length. The base is ideally a rectangle or modified rectangle such as a rectangle having rounded corners, and having a length that is from 2.5 to 5 times the length, and the corners can be square or rounded, and the material is sufficiently heavy to provide stability, such as 0.25-1.5 kilograms. It is further preferably that the base includes attachment member to hold it to a demonstration surface, and preferably a suction member, pressure clamp, such as a C-clamp, or magnetic connection. The base can also include recesses that hold sutures and posts supporting alligator clips for tendon material or artificial tendon simulations.

In a further embodiment the fixtures include the dome shaped member with the tissue sample that is used for the demonstration of a rotator cuff repair or a pair of dome-shaped members having a gap therebetween for a superior capsule procedure and for learning techniques in suturing and attachment of tissue specimens for shoulder surgery. It is also preferable that the modular system include a fixture that demonstrates the head of a humerus, including a bonnet shaped cuff tissue specimen that is attached on either side of the humeral head below the bony prominence. This fixture can also be provided with a bicipital groove and a ligament (also artificial) illustration for the purpose of illustrating the surgical technique.

It is also an advantage if the system includes a set of fixtures that together represent a knee joint. Thus, these two fixtures include a tibial component which extends upwards from a retainer in one end of the base member to provide the proximal tibia, including the tibial plateau positioned roughly orthogonal to the reference plane. There is also a distal femoral component, including at least the femoral condyles and the patellar notch. Ideally, the femoral fixture includes an extension or arm that extends upward from the retainer in the second distal portion of the base and forms a rim. The extension then forms a right angle so that the femoral condyles sit on the tibial plateau so as to mimic a knee positioned for knee surgery. The fixtures are sized so as to put the joint at an optimal position for viewing and learning a technique in the knee, including for example ACL repair or other minimally invasive surgical techniques.

In a further embodiment, the invention relates to a surgical demonstration system that includes one or more simulation tendons which are reinforced elastomeric members that replicates the look and feel, as well as the response to manipulation, of biological tendons, but which are artificial so as to allow a simulation for demonstration, training, sales and marketing of a surgical product that uses the tendon simulation to mimic biological tissue.

### BRIEF DESCRIPTION OF THE DRAWINGS

The present disclosure is best understood from the following detailed description when read with the accompanying Figures. It is emphasized that, in accordance with the standard practice in the industry, various features are not drawn to scale. In fact, the dimensions of the various features may be arbitrarily increased or reduced for clarity of discussion.

FIG. 1 illustrates a top view of an exemplary surgical demonstration and training system or kit;

FIG. 2 illustrates a side view of an exemplary surgical demonstration and training system;

FIG. 3 illustrates a top sectional view of an exemplary surgical demonstration and training system;

FIG. 4a illustrates a side view of an exemplary post for a simulated tendon graft procedure;

FIG. 4b illustrates a top view of the exemplary post of FIG. 4a;

FIG. 5 illustrates a side view of an exemplary joint fixture;

FIG. 6 illustrates a top view of an exemplary surgical demonstration and training soft tissue;

FIG. 7 illustrates a top view of an exemplary surgical demonstration and training soft tissue;

FIG. 8 illustrates an exemplary rotator cuff training fixture;

FIG. 9a illustrates a front view of an exemplary soft tissue support fixture;

FIG. 9b illustrates an exemplary side view of the soft tissue support fixture;

FIG. 9c illustrates a top view of exemplary soft tissue support fixture;

FIG. 10a illustrates a side view of an exemplary surgical demonstration and training system which includes a central fixture for a shoulder soft tissue repair and distal fixtures for knee procedures;

FIG. 10b illustrates a top and side view of an exemplary surgical demonstration and training system of FIG. 10a;

FIG. 10c illustrates a front top view of an exemplary surgical demonstration and training system of FIG. 10a;

FIG. 10d illustrates a back view of an exemplary surgical demonstration and training system of FIG. 10a;

FIG. 10e illustrates a bottom view of an exemplary surgical demonstration and training system of FIG. 10a;

FIG. 11a illustrates a posterior side view of an exemplary surgical demonstration and training system of a proximal humerus;

FIG. 11b illustrates an anterior side view of an exemplary surgical demonstration and training system of a proximal humerus;

FIG. 11c illustrates a medial side view of an exemplary surgical demonstration and training system of a proximal humerus.

FIG. 12a illustrates an artificial tendon simulation with an attached suture and needle;

FIG. 12b illustrates an artificial tendon simulation without the suture and needle;

FIG. 12c illustrates the reinforcement tape of the artificial tendon replacement of FIG. 12a, and;

FIG. 13 illustrates a detail of the reinforcement tape of the artificial tendon simulation of the present invention.

### DETAILED DESCRIPTION OF THE DRAWINGS

It is to be understood that the following disclosure describes several exemplary embodiments for implementing different features, structures, or functions of exemplary embodiments of the present invention. Exemplary embodiments of components, arrangements, and configurations are described below to simplify the present disclosure; however, these exemplary embodiments are provided merely as examples and are not intended to limit the scope of the invention. Additionally, the present disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the various Figures. Moreover, the formation of a first feature over or on a second feature in the description that follows may include embodiments in which the first and second features are formed in direct contact or as a unitary structure, and may also include embodiments in which additional features may be formed interposing the first and second features, such that the first and second features may not be in direct contact. Finally, the exemplary embodiments presented below may be combined in any combination of ways, i.e., any element from one exemplary embodiment may be used in any other exemplary embodiment, without departing from the scope of the disclosure.

Additionally, certain terms are used throughout the following description and claims to refer to particular components. As one skilled in the art will appreciate, various entities may refer to the same component by different names, and as such, the naming convention for the elements described herein is not intended to limit the scope of the invention, unless otherwise specifically defined herein. Further, the naming convention used herein is not intended to distinguish between components that differ in name but not function. Additionally, in the following discussion and in the claims, the terms “including” and “comprising” are used in an open-ended fashion, and thus should be interpreted to mean “including, but not limited to.” All numerical values in this disclosure may be exact or approximate values unless otherwise specifically stated. Accordingly, various embodiments of the disclosure may deviate from the numbers, values, and ranges disclosed herein without departing from the intended scope. Furthermore, as it is used in the claims or specification, the term “or” is intended to encompass both exclusive and inclusive cases, i.e., “A or B” is intended to be synonymous with “at least one of A and B,” unless otherwise expressly specified herein. Additionally, any directional terms, i.e., up, down, left, right, above, below, etc. used in the specification are not intended to be limiting upon any particular embodiment, as it is contemplated that any feature of the respective embodiments may be positioned in any location and is not limited to the particular location described in the exemplary embodiments herein. Further still, various shapes are described in this disclosure with respect to exemplary embodiments and the shapes are not intended to be limiting. For example, where a circle is described, the inventors contemplate that other shape such as ovals squares rectangles or triangles may also be used. As such, the shapes described in the following exemplary embodiments are not intended to be limiting as various shapes may be used without departing from the scope of the invention.

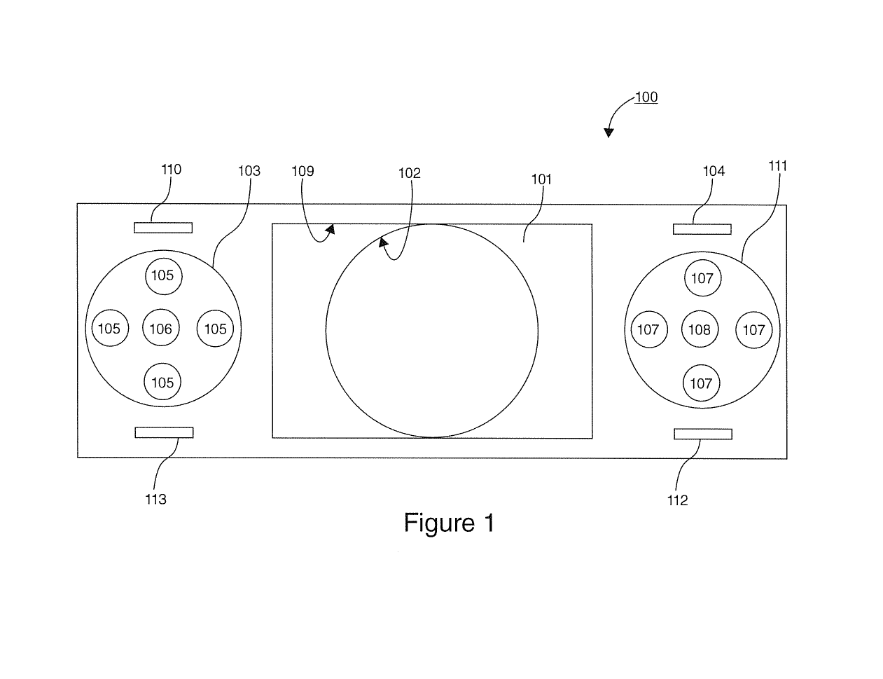

FIG. 1 illustrates a top view of an exemplary surgical demonstration system and training tool of the present disclosure. The surgical demonstration system and training tool generally includes a base member such as an elongated rectangular base manufactured from, for example, a plastic, nylon, vinyl, high impact polystyrene, polyethylene, polypropylene, acrylonitrile butadiene styrene (ABS), acrylic, or polycarbonate material. The base member is preferably sufficiently weighty to resist movement of the system during use and/or includes a restraining member, such as a clamp or suction arrangement. Embodiments of this disclosure are not limited to any particular material for forming the demonstration and training system base 100. The base may be injection molded or milled from a solid block of material. In one exemplary embodiment, the surgical demonstration system and training tool 100 includes a central fixture receptacle 101 that is configured to receive and secure surgical training fixtures therein. The fixture receptacle 101 is shown as being rectangular in the present exemplary embodiment, but the inventors contemplate that various other shapes (square, triangle, circular, trapezoidal, etc.) may be used without departing from the scope of the invention. The central rectangular fixture receptacle 101 is generally recessed into the training kit base 100, can including a central display area, and includes a center rectangular fixture recess wall 109 that surrounds the recess. The rectangular fixture wall 109 may be planar or it may have various frictional features, such as recesses or indentions formed therein, where the recesses or indentions formed therein may be used to engage a fixture positioned in the receptacle 101 to hold or secure the fixture in position, as will be further discussed herein. A central circular fixture receptacle 102 may also be recessed within the rectangular fixture receptacle 101. The recessed depths of the rectangular fixture receptacle 101 and the circular fixture receptacle 102 are generally of different depths.

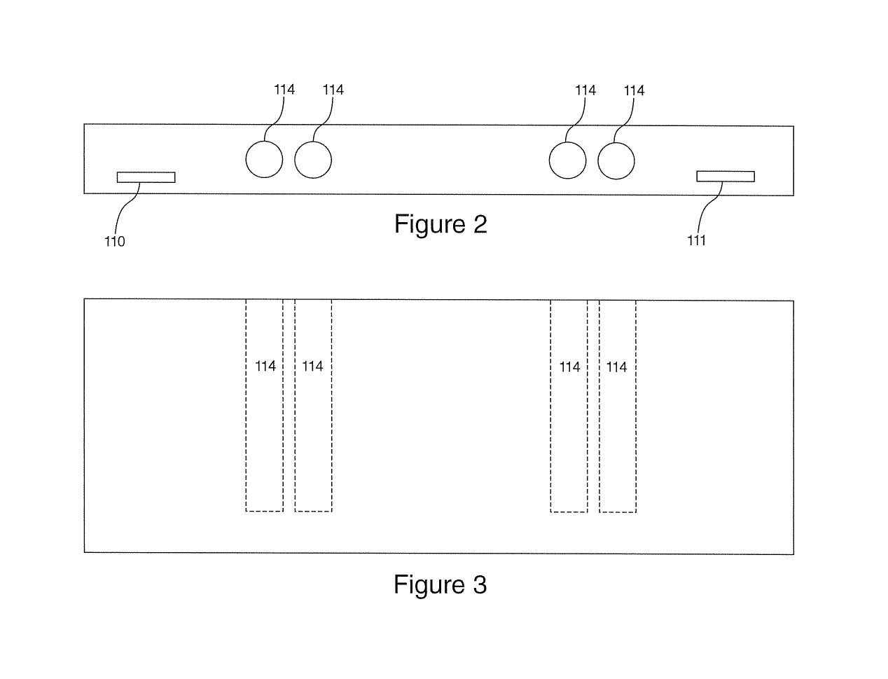

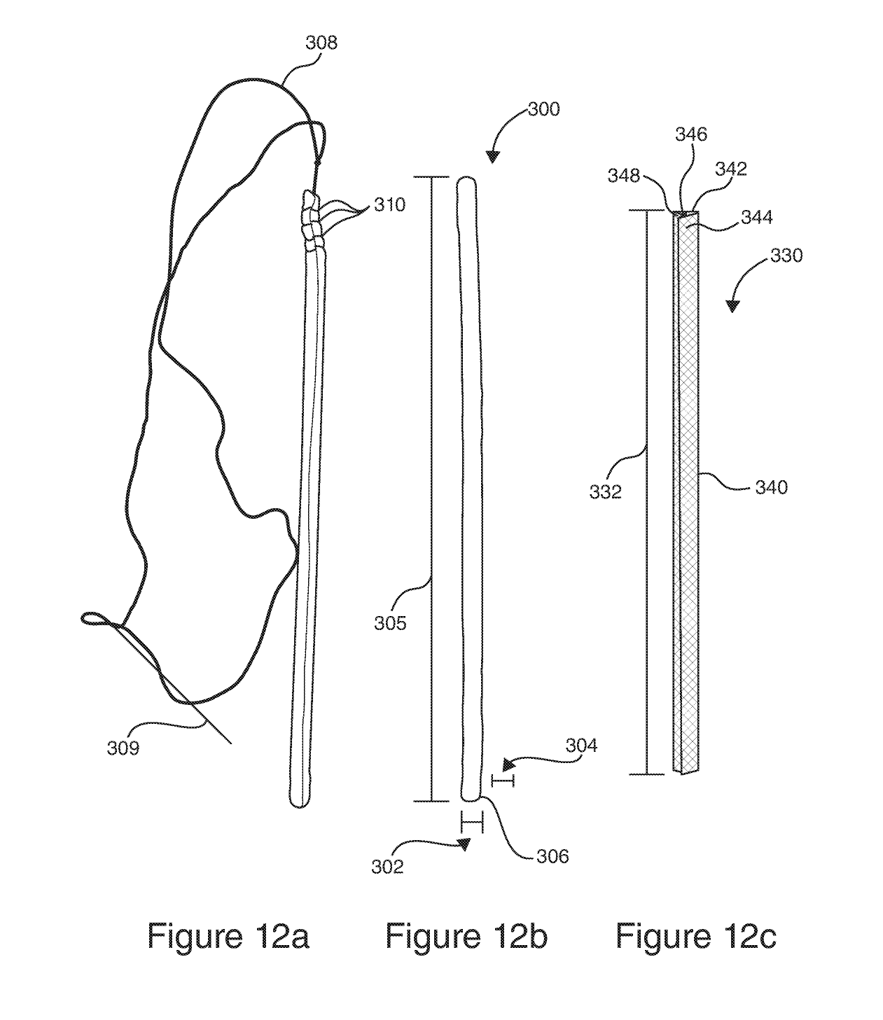

The distal ends of the rectangular fixture base 100 may each include additional fixture receptacles. For example, in FIG. 1 on the right side the exemplary training and demonstration fixture base 100 there may be an outer fixture receptacle area 104 that may or may not be recessed into the training base 100. In the current exemplary embodiment the outer fixture receptacle area 104 is shown as being circular, but embodiments of the disclosure contemplate that the receptacle area 104 may be any shape, such as square, rectangle, triangular, trapezoidal, oval, etc. The receptacle area 104 may include a plurality of receiving holes 107, 108, (which are shown as circular, but may also be square, oval, triangular, rectangular, trapezoidal, etc.) whereby the receiving holes may be positioned such that there are outer receiving holes 107 and a middle receiving hole 108. Similarly, on the left side of the exemplary training fixture base 100 there may be another outer fixture receptacle area 103 that may or may not be recessed into the training base 100. This outer fixture receptacle area 103 may also include a plurality of receiving holes 105, 106, whereby the receiving holes may be positioned such that there are outer receiving holes 105 and a middle receiving hole 106. The receiving holes permit the removable assembly of posts including engagement members, such as alligator clamps that hold simulated tendon material for demonstration and training purposes. The corners of the training base 100 may include a plurality of knot tying stations, 110, 111, 112, and 113. These knot tying stations generally include a rectangular or other shaped hole or passage formed into the base 100 that extends through from the top surface shown in FIG. 12 the side surface shown in FIG. 2. Therefore, surgical thread may be looped through the knot tying stations 110, 111, 112, and 113 and used to practice tying surgical thread knots to the fixture base 100 via the knot tying stations 110, 111, 112, and 113.

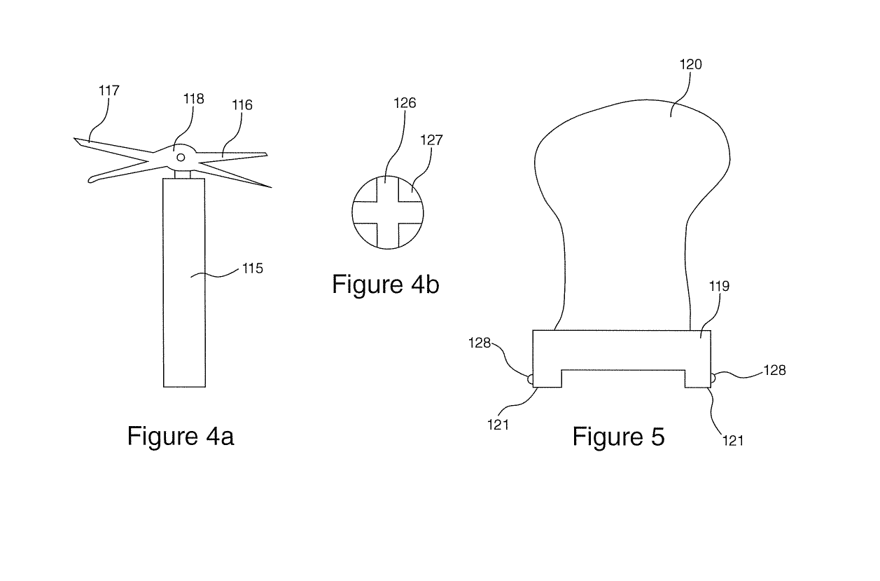

FIG. 2 illustrates a side view of the exemplary surgical demonstration system and training tool 100. The side of the demonstration and training system 100 includes a plurality of post storage holes 114 formed therein. These holes, which are also shown in a sectional view in FIG. 3, are sized in diameter, depth, and shape to receive the surgical training posts 115 therein. The surgical training posts 115 are further illustrated in FIGS. 4A and 4B. The posts 115 may generally be a cylindrical member having a top 127 configured to receive a securing device 118. The securing device may generally be an alligator type clip or other securing device configured to removably engage and secure a sample tendon or other practice tissue therein. Once used, the sample is replaced with a fresh sample for the next case. The exemplary securing device shown in FIG. 4a is an alligator clip having a pivotally movable handle 117 connected to pivotally movable jaws 116 that are configured to secure practice soft-tissue material therein. The securing device 118 is generally designed to be attached to the top surface 127 of the post 115. As such there may be various recesses or protrusions 126 formed into the top surface 127 of the post 115 that are configured to secure the clamping device 118 thereto.

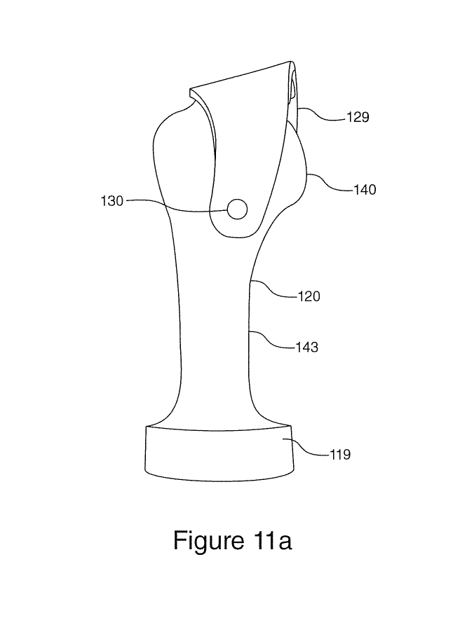

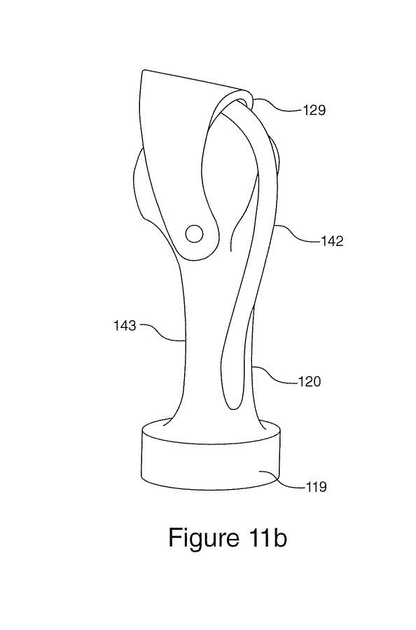

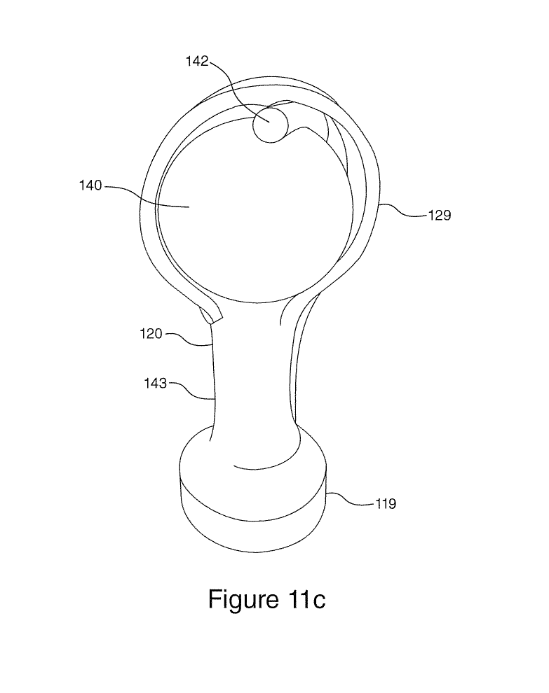

FIGS. 5 and 11a-c illustrate views of an exemplary joint fixture 120 which is a proximal humerus 140 that extends upward from the fixture base 119 from an arm member 143. The joint fixture 120 is provided with a soft tissue simulation, which in this case mimics a rotator cuff 129 and a bicipital tendon 142 may be used to practice various soft tissue repair procedures. As such, the joint fixture 120 is configured to be secured into the surgical demonstration system and training tool base 100. For example, the joint fixture 120 may be secured into one of the round fixture receptacles 103 & 104 of the surgical training and demonstration system base 100. As such, the fixture base 119 of the training fixture joint 120 may be sized to be received and secured within the recess of the fixture receiving areas 103 or 104. In alternative embodiments of the disclosure, the joint fixture may be received and secured into the center rectangular fixture recess 101. Regardless of the location where the fixture 120 is secured to the training and demonstration system 100, the lower portion of the surgical training fixture joint 120 may be frictionally engaged by the corresponding fixture recess wall to secure the surgical training fixture joint 120 to the surgical training system base 100. More particularly, the training fixture joint 120 may include terminating ends 121 on the base thereof that are configured to frictionally engage the fixture walls. Further still, the terminating ends 121 may also include a slight protrusion 128 formed thereon, whereby the slight protrusion 128 may form a snap lock with the rectangular fixture recess wall. The snap lock may be accomplished by forming a corresponding recess in the fixture wall that is configured to receive the protrusion 128 on the base of the training fixture 120, thus increasing the frictional engagement between the recess wall and the fixture 120. Further, embodiments of this disclosure contemplate various other means for increasing retention pressure on the various fixtures that may be inserted into the fixture receiving receptacles of the surgical training and demonstration kit base 100. The upper or top portion of the fixture joint 120 includes the bone or joint fixture shape that is to be used for the surgical technique practice. Various shapes for the fixture joint are contemplated within the scope of the present invention, and the invention is not limited to those shapes shown in the Figures. The size and shape of the fixture joints and training tissues discussed throughout this disclosure and are generally configured to approximate the training/demonstration anatomy size and shape. Further, the composition of the working portion of the fixture joints and other training/demonstration surfaces described herein is also configured to approximate the tissue/bone density properties or characteristics of the actual anatomy.

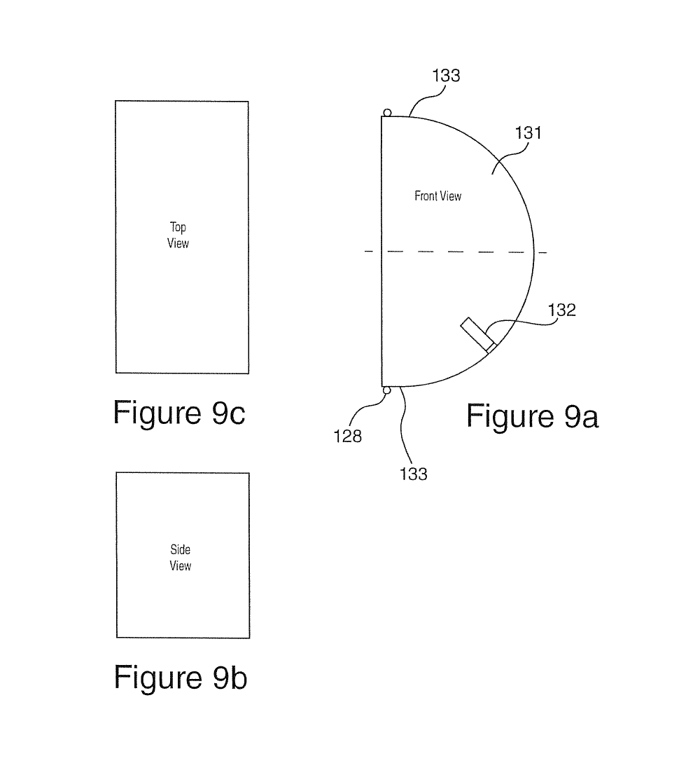

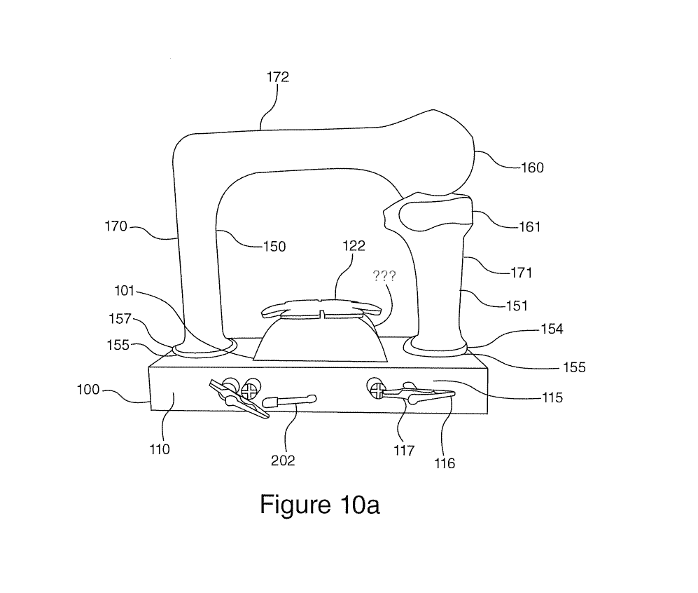

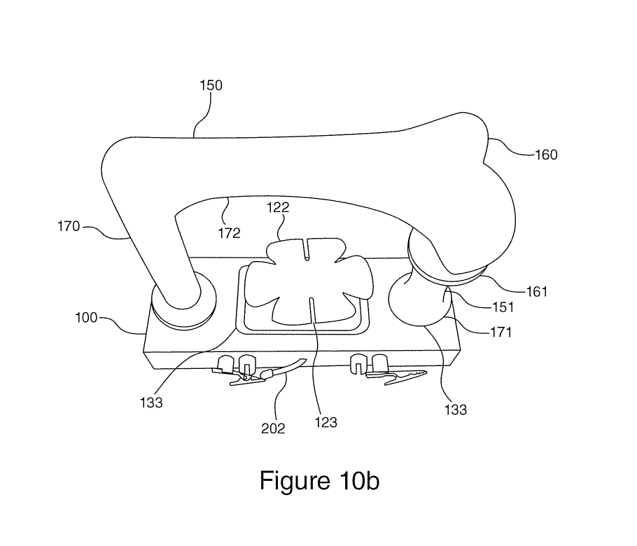

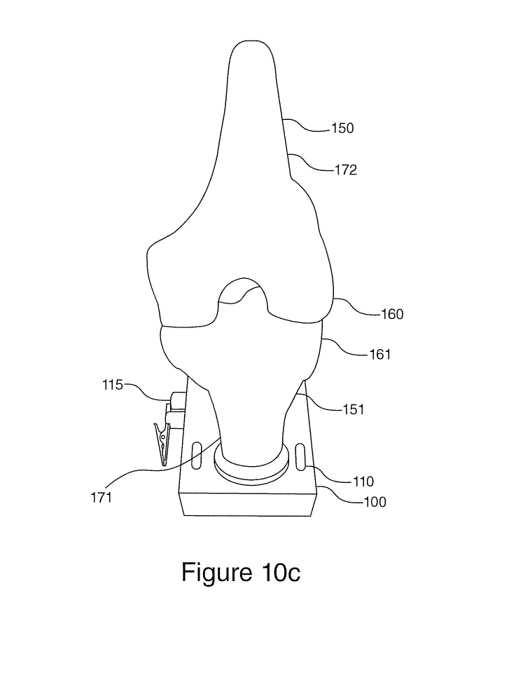

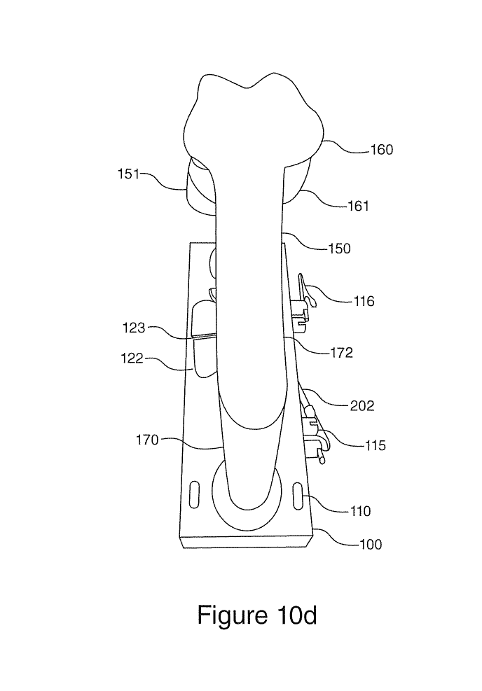

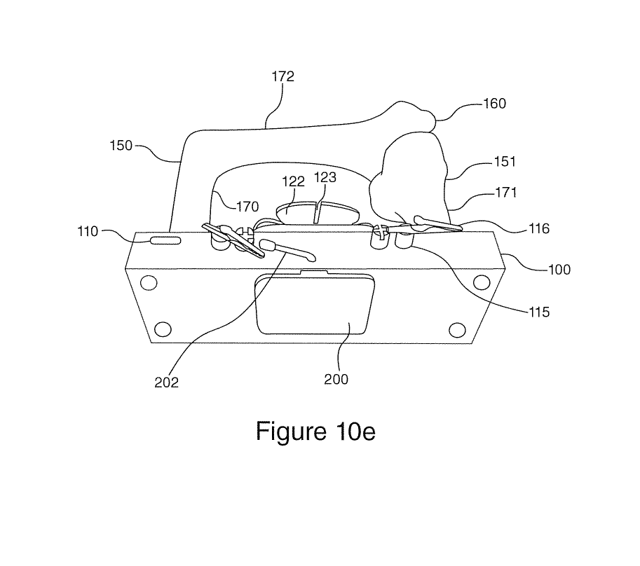

FIGS. 9a, b, and c illustrate three views of an exemplary fixture 131 that may be used in conjunction with the surgical training and demonstration system base 100. Specifically, FIG. 9a illustrates a frontal view of an exemplary soft-tissue support fixture 131. FIG. 9b illustrates a side view of the same fixture and FIG. 9c illustrates a top view of the same fixture. Generally speaking, the soft-tissue support fixture 131 includes a domed or semi-spherical surface that has a base portion 133 that is similar to the fixture base discussed with regard to FIG. 5. FIGS. 10a-10e illustrate a modified version of the soft tissue support which simulates a rotator cuff for the purposes of demonstrating rotator cuff repair. In this version 133′, the support is a domed member rather than a hemi-spherical member. The base portion 133′ will again be sized to be received with in the center rectangular fixture receptacle 101 of the surgical training kit base 100. The fixture 131 may be frictionally received within the receptacle 101. Further, fixture base 133 ‘may also include the snap lock members 128 as previously described. FIG. 9a also illustrates a suture anchor 132 embedded into the main body of the soft-tissue support fixture 131. The suture anchor 132 generally comprises a threaded rod that may be screwed into the surface of the fixture 131. The threaded rod may have a plurality of holes formed therethrough, whereby the holes are sized to receive surgical thread therethrough. Thus, when the suture anchor 132 is screwed into the support fixture 131, only the thread extends above the surface of the fixture. Thus, the thread may be used to practice surgical techniques in the same way as if the suture anchor 132 had been embedded in the patient's bone during an actual procedure.

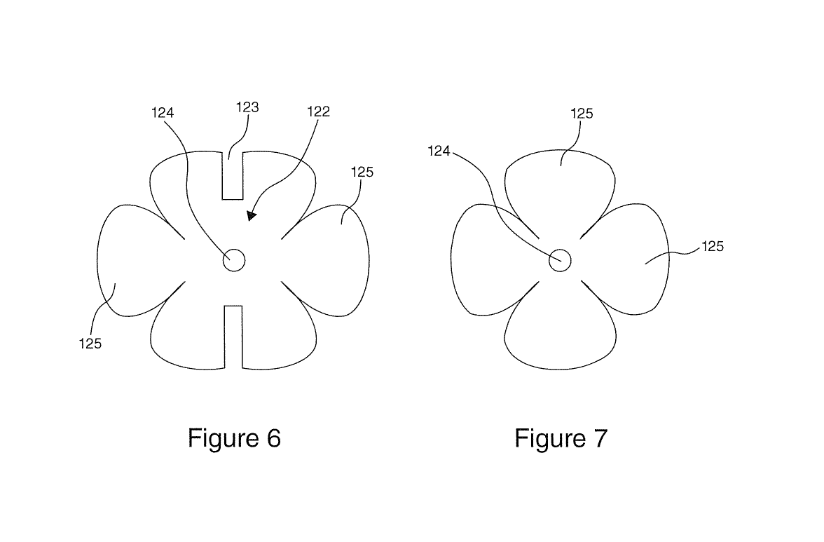

FIG. 6 illustrates a top view of an exemplary surgical training soft tissue 122 that may be used in the present disclosure. The soft tissue 122 may include a plurality of tissue flaps 125 and a slot cut 123 that operates to better approximate an actual tissue shape and characteristics of particular soft tissues in the human body. The surgical training tissue 122 may be attached to the soft-tissue support fixture 131 by a securing means that may use the central aperture 124 in the surgical training tissue 122. For example, a screw, securing post, post locking mechanism, or other means for securing the soft tissue 122 to the support fixture 131 may be used.

FIG. 7 illustrates a top view of another exemplary surgical training soft tissue that also has tissue flaps 125 and a central aperture through which training tissue may be secured to the soft-tissue support fixture 131.

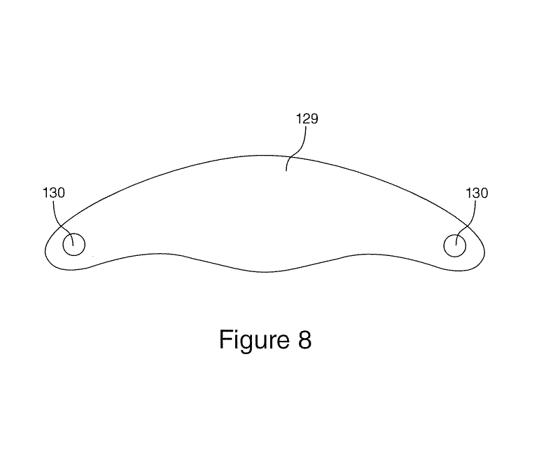

FIGS. 8 and 11a-c illustrate an further exemplary rotator cuff training fixture 129 that may be used to simulate surgical procedures on a rotator cuff by attaching the fixture 129 to the corresponding joint fixture (such as joint fixture 120 shown in FIG. 5). The rotator cuff fixture 129 includes distal ends having holes 130 formed there in that allow for attachment thereto. Embodiments of the present disclosure contemplate that various fixtures may be attached to the surgical training system base 100, wherein the various fixtures may simulate different anatomical parts of the body.

FIGS. 10a-e also illustrate a mating pair of fixtures 150, 151 which include the fixture base 152, 154 received in fixture recesses 155 formed in the base member. The fixtures include anatomical features which simulate the distal femur 160 at the end of one fixture 150 and a proximal tibia 161 at the end of the other mating fixture member 151. Advantageously as can be seen in FIGS. 10a-e, the pair of fixtures 150,151 are arranges so that the anatomical portions come together in the assembly to replicate a knee bent to 90° and positioned for knee surgery with the proximal tibial plateau positioned below the superior portion of the femoral condyles and exposing the maximum surfaces of the condyles. This is accomplished by providing the tibial fixture 151 with a vertical arm 171 that has a length corresponding to the length of the vertical arm 170 of the femoral fixture 150 to position the condyles above the plateau and the femoral fixture having an orthogonal arm 172 joined to the vertical arm 170 to position the condyles in over the plateau longitudinally. Other fixtures may include for example an entire skeletal foot or portions thereof.

In operation the surgical training demonstration system and training tool base 100 may be secured to a table or other fixed surface to facilitate surgical training or practice procedures, for example by the clamp or suction cup 200 having a handle for tightening and release 202. Other holding mechanisms may also be used, including for example, C-clamps, or magnets. The base 100 may be secured to the fixed table or other surface by one of many means, such as an external clamp that may engage the base 100 at the circular fixture receptacle 102 at one clamping point and an undersurface of the table or other fixed member at the second clamping point. Thus the clamp may be used to exert a force between the two clamping members that operates to secure the base 100 to the table. In other embodiments of the present disclosure various means of securing the base 100 to a table may be provided, for example, the underside of the base 100 may be fitted with one or more selectively actuated suction cups that may be mechanically activated to secure the base 100 to a table. Various other means for securing the base 100 to a table or otherfixed objects are contemplated within the scope of the present disclosure.

Once the base 100 is secured to a practice surface, various surgical techniques may be practiced, trained, or demonstrated. For example, a piece of surgical thread may be inserted through one of the knot tying locations 110-113 and pulled through the opposing side of the through hole. Then both ends of the thread may then be used to practice arthroscopic knot tying techniques.

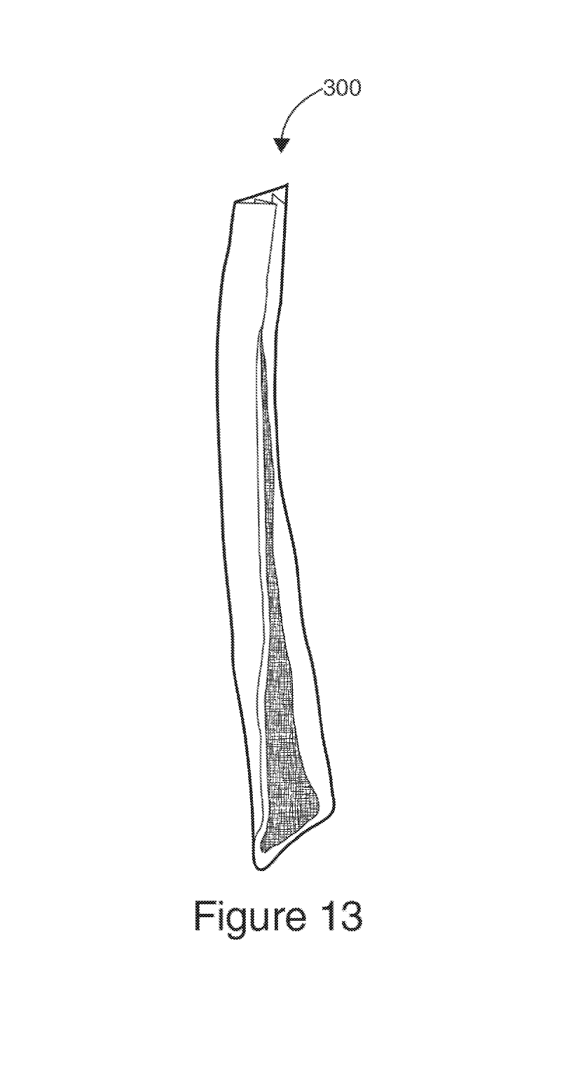

In a further embodiment of the invention, FIGS. 12 a-12c, and FIG. 13 relate to an artificial tendon simulation that is intended for demonstration purposes, and not for implantation or use within the body. Thus, there are considerations outside of the biological context which come into consideration, for example, delamination during use becomes a problem, durability and shelf life over a wider variety of temperatures, humidities and shipping and storage conditions, and the aesthetics. It is not necessary for an implant to retain a natural look, in particular after it is implanted, but a demonstration simulation needs to look and feel natural throughout it's useful life.

In particular, FIG. 12b illustrates the tendon simulation 300 which has the look and feel of a human tendon. This tendon is a ribbon-like shape (i.e. a long rectangle having a width or diameter across 302, a depth 304 and a length 305 and which can include rounded corners 306). The tendons are shaped and sized for use in the demonstration and training of particular surgical techniques. The shape and size is thus dependent on the technique or implant that is being demonstrated, and a list follows below with the tendon name, width or diameter, and length listed, which can vary in diameter between 5 and 15 mm in diameter, or for tendon sheaths between 0.5 and 3 mm, by 20 to 40 mm and in length from 50 to 250 mm. In some ispecific instances, the tendon 300 is approximately 25-30 centimeters long, 0.5-10 centimeter wide, (and 0.5-8 millimeters deep), and more preferably 7+/−2 millimeters in diameter, and if flat or rectangular 0.5-2.5 millimeters in thickness). FIG. 12a illustrates the tendon having a suture 308 and surgical needle 309 connected by a whip stitch 310 at one end.

FIG. 12c and FIG. 13 illustrate the woven fabric (preferably polyester or nylon) substrate 330 which underlies an elastomeric coating. The fabric reinforcement has a length 332 that is substantially the same as the final product minus the coating layer, which is 0.01-0.25 mm in thickness, and preferably from 0.05 to 0.1 mm, and more preferably 0.08 +/−0.025 mm. The fabric substrate is preferably a folded bias weave as is shown in FIG. 13, and comprises a center v-fold 340 having a first outer surface 342 opposing a second outer surface 344 each of which terminate in a top fold 346, 348 which are folded inward toward the center v-fold 340. After coating, the top folds oppose each other so that the substrate has a rectangular shape represented by the first and second outer surface with an inner construction comprising the two opposing top folds. This construction provides the restraint to elongation that a natural tendon has even though the coating is an elastomer and gives the impression of elasticity.

The tendon has a feel that is elastomeric with a slight tack, but in fact, has very little elongation along the length of the tendon. In fact, the proportions and the shapes can be varied in order to simulate specific tendons and to enable the look and feel of tendons for the purpose of specific surgical procedures. The proportions of specific tendon simulations are provided below with the tendon that the artificial simulation is intended to replicate. In some instances, the artificial tendons are also provided with simulation “bone plugs, which are attached artificial polymeric material meant to simulate bone. In these cases, the tendon simulations are inserted into the bone plugs. Where there is a diameter indicated, the tendon replacements are more cylindrical in shape, having a round or rounded cross-section (i.e. circular or ovoid), rather than the tape-like shape of the tendon simulations with thickness given.

In practice, the surgeries that repair or replace tendon, including for example, ACL reconstructions, using for example, continuous loop graft, quad tendon harvest for ACL reconstruction, various doubled, tripled or quadrupled looped graft techniques, tissue anchoring techniques, and both tendon harvesting and allograft techniques, require practice to perfect. Issues that the surgeons encounter include issues manipulating the instruments, including needles and needle holders, suture control, including knot tying, winding, keeping them from tangling, and proper tensioning. The grafts are often threaded through bone tunnels or into tight locations within a joint. In addition, natural tendons are tacky and tend to adhere to each other. It is an extreme advantage for a surgeon or assistant learning the technique or to assist for a technique to have had sufficient time to practice working with a tendon simulation that reacts in the same way that a tendon will. This means that the tendon should present the same issues that a natural tendon will, including shearing (but not delaminating the coating from the substrate especially during a bone tunneling technique that requires a tendon to be threaded through a bone tunnel), and reaction to cutting suturing like a natural tendon, which again suggests that the coating should not delaminate at the edges or corners. Advantageously, the tendon will have the same feel and reaction to manipulation as a natural tendon since part of the technique requires that the graft is under the proper tension when the surgery is finished and the joint is extended. This aspect of joint reconstruction often requires quite a bit of practice to understand how much tendon needs to be used for a particular surgery. The present invention allows the surgical team to perfect the surgical technique in a manner in which the surgeon has clear vision of the hard and soft tissues, as well as the instruments and the surgical implements (including the sutures, tissue anchors, and needles).

The dimensions for application specific examples are given below:

### Bone—Patellar Tendon—Bone Grafts

8 mm diameter×20 mm, 9 mm×20 mm & 10 mm×20 mm bone plugs−Total lengths for each option 85 mm, 90 mm, 95 mm & 100 mm

### Quad Tendon/Soft Tissue Only

8 mm, 9 mm & 10 mm diameter options and 70 mm, 75 mm & 80 mm length options
Quad Tendon with Patellar Bone Plug/Dowell
8 mm, 9 mm & 10 mm diameter options and 70 mm, 75 mm & 80 mm length options

### Hamstring Tendons

SemiTendonosis
Lengths=170 mm, 180 mm, 190 mm, 200 mm, 210 mm, 220 mm, 230 mm & 240 mm
Diameters of 7 mm, 7.5 mm, 8 mm, 8.5 mm, 9 mm, 9.5 mm & 10 mm

Gracilis
Lengths=170 mm, 180 mm, 190 mm, 200 mm, 210 mm, 220 mm
Diameters of 4 mm, 4.5 mm, 5 mm, 5.5 mm, 6 mm, 6.5 mm, 7 mm, 7.5 mm, 8 mm, 8.5 mm
   Achilles with Bone Plug/Dowell

Diameters=8 mm-12 mm

Lengths=160 mm 270 mm

### Achilles Soft Tissue Only

Diameters=8 mm-12 mm

Lengths=160 mm 270 mm

### Anterior Tibialis Tendon

Lengths=170 mm, 180 mm, 190 mm, 200 mm, 210 mm, 220 mm, 230 mm, 240 mm, 250 mm, 260 mm, 270 mm
Diameters of 7 mm, 7.5 mm, 8 mm, 8.5 mm, 9 mm, 9.5 mm, 10 mm, 10.5 mm, 11 mm

### Posterior Tibialis Tendon

Lengths=170 mm, 180 mm, 190 mm, 200 mm, 210 mm, 220 mm, 230 mm, 240 mm, 250 mm, 260 mm, 270 mm
Diameters of 7 mm, 7.5 mm, 8 mm, 8.5 mm, 9 mm, 9.5 mm, 10 mm, 10.5 mm, 11 mm

### Peroneus Longus Tendon

Lengths=170 mm, 180 mm, 190 mm, 200 mm, 210 mm, 220 mm, 230 mm, 240 mm, 250 mm, 260 mm, 270 mm
Diameters of 7 mm, 7.5 mm, 8 mm, 8.5 mm, 9 mm, 9.5 mm, 10 mm, 10.5 mm, 11 mm

### Palmaris Tendon

Lengths=50 mm-120 mm

Diameter=3 mm-4.5 mm

### Palmaris Slip Tendon Harvest

Lengths=50 mm-120 mm

Diameter=1.5 mm-4 mm

### Demonstration/Simulation Dermal and Tissue Graft Patches & Jackets

These range is size from 0.5 mm thick to 4 mm+thick and are typically procured in rectangular, square or circular form

Hand/Wrist Tendon Sheath Augmentation sizes
(30 mm×40 mm×0.5 mm) (40 mm×40 mm×1.0 mm)

### Achilles Tendon Augmentation

(40 mm×70 mm×1.0 mm) (40 mm×70 mm×1.5 mm) (50 mm×90 mm×1.5 mm) (40 mm×70 mm×2.0 mm)

### Peroneal/Tibial Tendon Augmentation

(40 mm×70 mm×1.0 mm) (40 mm×70 mm×1.5 mm) (15 mm×140 mm×1.5 mm)

### Rotator Cuff Augmentation

(40 mm×40 mm×1.0 mm) (35 mm×35 mm×1.5 mm) (35 mm×35 mm×2.0 mm) (40 mm×70 mm×3.0 mm)

Preferably the tendon simulation is constructed of an elastomeric material that totally envelopes a fiber reinforcement. Advantageously, the fiber reinforcement can be a bias cut material. In the case of the long cylinder or tape shaped tendon simulations, the reinforcement material is likewise a ribbon or tape shape, and is advantageously folded, or even a triple fold resulting in a four layer fabric substrate having opposing inner-most layers joined at the top to out layers forming a v-shape with a center fold. The fabric is preferably a bias weave with an open construction to allow for penetration and a suitable resistance to elongation along the length of the tendon simulation. The textile may be polyester, nylon, cotton, or wool or other suitable natural or artificial fibers or blends thereof.

The outer material is an elastomer, such as an elastomer of silicone, or a carbon based polymer, including for example, EVA, latex, chloroprene, butyl rubber or vinyl chloride, with a preferred material being an Ecoflex super soft addition cure silicon rubber, such as Ecoflex 00-20 (sold by Smoothon with a Shore A hardness of 00-20 in accordance with ASTM D-2240 and a tensile Strength of 160 psi in accordance with ASTM D-412, a 100% modules of 8 psi in accordance with ASTM d-412, an elongation at Break % of 845% in accordance with ASTM 412, and a Die and Tear Strength of 30 PLI in accordance with ASTM D-624. Other materials with characteristics of +/−25%, preferably +/−20%, and more preferably +/−15 or even 10%, can be substituted for this particular silicone rubber. It is preferable that the elastomer has a tack, a soft feel, and a white color, as natural tendons do. A further suitable condensation sure silicone elastomer is Bluesil V1082, sold by Bluestar Silicones, which has a Shore A hardness of 9, a tensile of 480, an Elongation to Break of 570, and a tear of 110. If the elastomer is not white, a colorant or pigment, such as titanium dioxide, can be added in an appropriate amount to produce a white, opaque tendon which has a slight lucidity and even a slight sheen on the surface, like a natural tendon. The closer the appearance to a natural tendon, the better, and the longer the appearance is sustained under conditions including shipping, storage, exposure to sunlight and office or operating room light conditions, the better for the present purpose.

In addition, it is preferable that the tendon simulation includes an adhesion promoting primer or adhesive between the reinforcing tape and the elastomeric layers. A suitable adhesive is Bluesil V04 which is a dilute solution of a reactive silicone resin in naphtha used as an adhesion promoter.

The tendon simulations can be made by applying the adhesion promoter to the fiber reinforcement, and placing that within a mold, such an an injection mold, and inserting the silicone elastomer in the mold so as to envelope the reinforcing material by at least 0.05 mm, and preferably by at least 0.1 and more preferably by 0.25-3 mm, and subsequently allowing the elastomer to cure about the reinforcing material. In the event an artificial bone plug is supplied it can be molded or cast on an end of the tendon simulation, for example, by molding a rigid polymeric cylinder, having a diameter of from 5 to 15 mm, and a length of from 5 to 25 mm, which could be a polystyrene or polyester, phenolic resin for example. In certain surgical techniques, the bone plugs are used to anchor a tendon replacement in a surgically constructed tunnel by wedging the plug into a leading edge of the tunnel.

In another embodiment of the present disclosure the surgical demonstration system and training tool may be used to practice, train, or demonstrate soft tissue repair techniques. To do this the surgical training and demonstration system base 100 is again affixed to a practice surface via a clamp or other securing means. Then a fixture, such as the soft-tissue support fixture 131, is attached to the surgical training and demonstration system training base 100 at the center rectangular fixture receptacle 101. Then one or more surgical training tissue samples 122 may be attached to the top surface of the soft tissue support fixture 131 through a screw, snap mechanism, or other securing means that will generally go through the central aperture 124 of the training tissue 122 into the top of the soft tissue support fixture 131. Once the training tissue 122 is secured to the support fixture 131, a suture anchor 132 having one or more surgical threads attached thereto may be screwed into or otherwise embedded into the surface of the soft tissue support fixture 131 at a position below one of the tissue flaps 125 of the surgical training tissue 122.

Once the surgical training tissue 122 and the suture anchor 132 are positioned on the soft-tissue support fixture 131, the soft tissue repair technique may be practiced. Specifically, an anchor hole is drilled into the fixture, and then the threads attached to the suture anchor 132 may be threaded through the tissue flap 125 using a suture needle and then surgically knotted so as to attach the flap 125 of surgical training tissue 122 to the soft tissue support fixture 131. Since each of the surgical training tissues 122 generally contain a plurality of tissue flaps 125, multiple suture anchors 132 may be inserted into the tissue support fixture 131 to practice securing each of the tissue flaps 125 to the tissue support fixture 131. After multiple suture anchors 132 are inserted into the tissue support fixture 131, it may be necessary to replace the tissue support fixture 131 to provide a new and unused surface for insertion of additional suture anchors 132.

In another embodiment of the present disclosure the surgical demonstration system and training tool may be used to practice soft-tissue repair on a joint. For example, the surgical training fixture joint 120 may be secured to the surgical demonstration system and training tool base 100. Thereafter, a piece of surgical training tissue 122 that may be sized and shaped to mimic the tissue corresponding to the actual joint may be attached to the fixture 120 by a screw or other attachment mechanism. Thereafter, one or more suture anchors 132 may be inserted into the fixture 120, and each of the suture anchors 132 may have one or more surgical threads extending therefrom. The surgical threads may be sutured to the training tissue and knotted so that the tissue is positioned appropriately on the joint as to mimic a surgical repair of the joint tissue. During the training repair process, sample tendons and other soft-tissue may also be inserted under the surgical training tissue 122 so as to accurately depict the training environment and the space constraints associated with the actual procedure.

In another embodiment of the present disclosure, the surgical training and demonstration system base 100 may be used to simulate an ACL graft preparation procedure. Specifically, the training system base 100 may be secured to a table and then one of the posts 115 may be inserted into any one of the holes 105, 106, 107, 108 formed into the base 100. The clamping mechanism 118 may be secured to the upper surface 127 of the post 115. Then a practice or sample tendon specimen may be clamped into the clamping mechanism 118 and the distal end thereof may be extended from the clamping mechanism and held for the surgical procedure. The distal end of the sample tendon may then be sutured a number of times to simulate ACL graft preparation, while the opposite end of the sutures is secured and held steady by the clamp 118 and post 115 combination to simulate the actual surgical procedure where the tendon is attached to the patient.

In another embodiment posts 115 may be positioned on opposing ends of the training base 100, i.e. one in the left receptacle area 103 and one in the right receptacle area 104. Each of the posts 115 may be fitted with a clamp 118 and each may have their own sample tendon specimen secured thereto. The opposing ends of each of the tendon specimen may be pulled together near the middle of the demonstration system and training tool base 100 and surgically sutured together to practice the tendon repair technique. In this embodiment the base operates to hold in a position that simulates the actual surgical procedure.

In each of the embodiments where the posts 115 are used, once the training procedure has completed, each of the posts 115 may be inserted into the post storage holes 114 formed into the side of the surgical demonstration system and training tool base 100. This allows for convenient storage of the posts 115 integral with the surgical training kit base 100.

In another embodiment of the present disclosure, the surgical demonstration system and training tool may be used to simulate soft tissue repair that spans an open space between to harder surfaces, i.e., bones or joints. In this embodiment the upper hemispherical shaped soft tissue support fixture 131, for example, may be divided in half and each of the respective halves may be secured to the base 100 in the rectangular fixture receptacle 101. The two halves of the support fixture 131 may be positioned such that there is a gap between the two fixture portions. Then a soft tissue 122 may be laid over the top of the two fixture halves such that the soft-tissue 122 spans the gap between the two fixture halves. Then the suture anchors 132 may be positioned in each of the respective fixture halves and the accompanying suture thread may be used to secure the training tissue 122 to the respective fixture halves, thus simulating the tissue repair between two bones or joints.

The foregoing has outlined features of several embodiments so that those skilled in the art may better understand the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. A surgical training and demonstration tool, comprising:
a base member comprising:
a first fixture receptacle having a recessed portion defined by recess wall; and
a surgical training fixture comprising:
a fixture base member sized to be frictionally received in the recessed portion of the first fixture receptacle and a tendon simulation retaining member; and
a tendon simulation member comprising a woven reinforcement member enveloped in an elastomer having a Shore A hardness of 00-20 in accordance with ASTM D-2240 and a tensile Strength of 75-200 psi in accordance with ASTM D-412

2. The surgical training tool of claim 1, wherein the surgical training fixture is a post and the tendon simulation retaining member is a clamping device attached to a top surface of the posts, the clamping device being configured to clamp and secure the tendon simulation member during a surgical demonstration.

3. The surgical training tool of claim 2, wherein the base further comprises a plurality of post receiving holes formed into the side surface, the post receiving holes being sized to receive one or more of the posts for storage.

4. The surgical training tool of claim 3, further comprising at least one knot tying station comprising a through hole formed between a top and side surface of the base member.

5. The surgical training tool of claim 1, wherein the tendon simulation member has a ribbon-like or cylindrical shape of from 10 to 30 centimeters long, from 0.5-10 centimeter wide, and 1-2.5 millimeters in thickness and has an elongation along the length of the tendon of less than 10%.

6. The surgical training tool of claim 1, wherein the tendon simulation member comprises an elastomer which envelopes a woven textile reinforcement layer.

7. The surgical training tool of claim 6, wherein the elastomer is a silicone elastomer.

8. The surgical training tool of claim 7, further comprising an adhesion promoting layer between the textile reinforcement layer and the elastomer.

9. The surgical training tool of claim 6, wherein the textile reinforcement layer comprises from 0.3 to 0.8 of the thickness of the tendon simulation member.

10. The surgical training tool of claim 6, wherein the textile reinforcement layer comprises from 0.4 to 0.6 of the thickness of the tendon simulation member.

11. The surgical training tool of claim 6, wherein the textile reinforcement layer comprises a folded bias weave fabric.

12. The surgical training tool of claim 11, wherein the textile reinforcement layer comprises a V-shape fold.

13. The surgical training tool of claim 12, wherein the textile reinforcement layer comprises a triple fold v-shape in which the outer edges fold inward to the center fold.

14. A surgical training and demonstration tool system comprising:
a base member having a tissue simulation retaining member; and
a tissue simulation member comprising a woven reinforcement member enveloped in an elastomer having a Shore A hardness of 00-20 in accordance with ASTM D-2240 and a tensile Strength of 75-200 psi in accordance with ASTM D-412.

15. The surgical training tool of claim 14, wherein the tissue simulation member has a ribbon-like or cylindrical shape of from 10 to 30 centimeters long, from 0.5-10 centimeter wide, and 1-2.5 millimeters in thickness and has an elongation along the length of the tendon of less than 10%.

16. The surgical training tool system of claim 14, wherein the tissue simulation member comprises an elastomer which envelopes a woven textile reinforcement layer.

17. The surgical training tool system of claim 16, wherein the elastomer is a silicone elastomer.

18. The surgical training tool system of claim 17, further comprising an adhesion promoting layer between the textile reinforcement layer and the elastomer.

19. The surgical training tool system of claim 16 wherein the textile reinforcement layer comprises from 0.3 to 0.8 of the thickness of the tissue simulation member.

20. The surgical training tool system of claim 16, wherein the textile reinforcement layer comprises from 0.4 to 0.6 of the thickness of the tissue simulation member.

21. The surgical training tool system of claim 16, wherein the textile reinforcement layer comprises a folded bias weave fabric.

22. The surgical training tool system of claim 21, wherein the textile reinforcement layer comprises a v-shape fold.

23. The surgical training tool system of claim 22, wherein the textile reinforcement layer comprises a triple fold v-shape in which the outer edges fold inward to the center fold.

24. The surgical training tool system of claim 22, wherein the tissue simulation member is one or more of a tendon selected from the group comprising a Patellar Tendon having 5-10 mm diameter and with a length of 75-120 mm; a Quad Tendon having 5-10 mm diameter and a length of 60-90 mm, a SemiTendonosis Tendon having a diameter of 5-12 mm and a length of 150-300 mm; an Anterior Tibialis Tendon having a diameter of 6-12 mm and a length of 150-300 mm; a Posterior Tibialis Tendon having a diameter of 5-12 mm and a length of 150-300 mm; a Peroneus Longus Tendon having a diameter of 5-12 mm and a length of 150-300 mm; a Palmaris Tendon having a diameter of 2-5 mm and a length of 20-120 mm and a harvest Palmaris Slip Tendon a diameter of 1-5 mm and a length of 50-120 mm.

25. The surgical training tool system of claim 24, wherein the soft tissue simulation member includes a bone simulation member connected thereto having a length of 2-12 mm and a diameter of 3-20 mm.
